SEQUENCE LISTING (68 sequences)

SEQ ID NO: 1 - protein, 177 aa, Homo sapiens
MACCQTSFCGFPSCSTSGTCGSSCCQPSCCETSSCQPRCCETSCCQPSCCQTSFCGFPSFSTGGTCDSSCCQPSCCETSCCQPSCYQTSSCGTGCGIGGGIGYGQEGSSGAVSTRIRWCRPDCRVEGTCLPPCCVVSCTPPSCCQLHHAEASCCRPSYCGQSCCRPVCCCYCSEPTC

SEQ ID NO: 2 - protein, 167 aa, Homo sapiens
MTCCQTSFCGYPSCSTSGTCGSSCCQPSCCETSCCQPSCCQTSFCGFPSFSTSGTCSSSCCQPSCCETSCCQPSCCQTSSCGTGCGIGGGIGYGQEGSSGAVSTRIRWCRPDCRVEGTCLPPCCVVSCTPPTCCQLHHAEASCCRPSYCGQSCCRPVCCCYSCEPTC

SEQ ID NO: 3 - protein, 174 aa, Homo sapiens
MTCCQTSFCGYPSFSISGTCGSSCCQPSCCETSCCQPRSCQTSFCGFPSFSTSGTCSSSCCQPSCCETSCCQPSCCETSCCQPSCCQISSCGTGCGIGGGISYGQEGSSGAVSTRIRWCRPDSRVEGTYLPPCCVVSCTPPSCCQLHHAQASCCRPSYCGQSCCRPVCCCEPTC

SEQ ID NO: 4 - protein, 131 aa, Homo sapiens
MACCQTSFCGFRSFSTGGTCDSSCCQPSCCETSCCQPSCYQTSSCGTGCGIGGGIGYGQEGSSGAVSTRIRWCRPDCRVEGTCLPPCCVVSCTPPSCCQLHHAEASCCRPSYCGQSCCRPVCCCYCSEPTC

SEQ ID NO: 5 - protein, 85 aa, Homo sapiens
MACCQTSFCGFPSCSTSGAVSTRIRWCRPDCRVEGTCLPPCCVVSCTPPSCCQLHHAEASCCRPSYCGQSCCRPVCCCYCSEPTC

SEQ ID NO: 6 - protein, 177 aa, Homo sapiens [sequence omitted]

SEQ ID NO: 7 - protein, 141 aa, Homo sapiens
MTCCQTSFCGYPSCSTSGTCGSSCCQPSCCETSCCQPSCCETSCCQPSCCQTSSCGTGCGIGGGIGYGQEGSSGAVSTRIRWCRPDCRVEGTCLPPCCVVSCTPPTCCQLHHAEASCCRPSYCGQSCCRPVCCCYSCEPTC

SEQ ID NO: 8 - protein, 96 aa, Homo sapiens
PVTCQTTVCRPVTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 9 - protein, 33 aa, Homo sapiens
TTCCQPVSVQSPCCRPPCGQPTPCSTTCRTSSC

SEQ ID NO: 10 - protein, 85 aa, Homo sapiens
VTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 11 - protein, 128 aa, Homo sapiens
MTGSCCGSTLSSLSYGGGCCQPCCCRDPCCCRPVTCQTTVCRPVTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 12 - protein, 98 aa, Homo sapiens
MYCCALRSCSVPTGPATTFCSFDKSCRCGVCLPSTCPHEISLLQPICCDTCPPPCCKPDTYVPTCWLLNNCHPTPGLSGINLTTYVQPGCESPCEPRC

SEQ ID NO: 13 - protein, 98 aa, Homo sapiens
MDCCASRSCSVPTGPATTICSSDKSCRCGVCLPSTCPHTVWLLEPICCDNCPPPCHIPQPCVPTCFLLNSCQPTPGLETLNLTTFTQPCCEPCLPRGC

SEQ ID NO: 14 - protein, 98 aa, Homo sapiens
MDCCASRGCSVPTGPATTICSSDKSCRCGVCLPSTCPHTVWLLEPTCCDNCPPPCHIPQPCVPTCFLLNSCQPTPGLETLNLTTFTQPCCEPCLPRGC

SEQ ID NO: 15 - protein, 136 aa, Homo sapiens
MVNSCCGSVCSDQGCGLENCCRPSCCQTTCCRTTCCRPSCCVSSCCRPQCCQSVCCQPTCCSPSCCQTTCCRTTCCRPSCCVSSCFRPQCCQSVCCQPTCCRPSCGQTTCCRTTCYRPSCCVSTCCRPTCSSGSCC

SEQ ID NO: 16 - protein, 98 aa, Homo sapiens
TCCRPSCCISSCCRPSCCISSCCKPSCCQTTCCRPSCCISSCYRPQCCQPSCCRPACCISSCCHPSCCVSSCRCPFSCPTTCCRTTCFHPICCGSSCC

SEQ ID NO: 17 - protein, 166 aa, Homo sapiens
MVNSCCGSVCSDQGCGLENCCRPSYCQTTCCRTTCCRPSCCVSSCCRPQCCQTTCCRTTCCHPSCCVSSCCRPQCCQSVCCQPTCCRPQCCQTTCCRTTCCRPSCCRPQCCQSVCCQPTCCCPSYCVSSCCRPQCCQTTCCRTTCCRPSCCVSRCYRPHCGQSLCC

SEQ ID NO: 18 - protein, 186 aa, Homo sapiens
MVSSCCGSVSSEQSCGLENCCCPSCCQTTCCRTTCCRPSCCKPQCCQSVCYQPTCCHPSCCISSCCRPYCCESSCCRPCCCRPSCCQTTCCRTTCCRTTCCCPSCCVSSCCRPQCCQSVCCQPTCCRPSYCISSCCHPSCCESSCCRPCCCVRPVCGRVSCHTTCYRPTCVISTCPRPLCCASSCC

SEQ ID NO: 19 - protein, 109 aa, Homo sapiens
SPLAAVPAAASPAAVAPAAVCPGAAGPSAASLCAASQPAAVPAAASPAAAAPLAVNPAAAARAAAAPAAACVQSVAESPATPLAIAQPVSFPPAPVPCAVPPLAAELLA

SEQ ID NO: 20 - protein, 210 aa, Homo sapiens
MVSSCCGSVCSDQGCGQVLCQETCCRPSCCQTTCCRTTCYRPSCCVSSCCRPQCCQSVCCQPTCCRPSCCETTCCHPRCCISSCCRPSCCMSSCCKPQCCQSVCCQPTCCHPSCCISSCCRPSCCVSRCCRPQCCQSVCCQPTCCRPSCCISSCCRPSCCESSCCRPCCCRPCCCLRPVCGRVSCHTTCYRPTCVISTCPRPLCCASSCC

SEQ ID NO: 21 - protein, 114 aa, Homo sapiens
SSCCKPQCCQSVCCQPTCCHPSCCISSCCRPSCCVSSCCKPQCCQSVCCQPTCCHPSCSISSCCRPSCCESSCCRPCCCVRPVCGRVSCHTTCYRPTCVISTCPRPVCCASSCC

SEQ ID NO: 22 - protein, 191 aa, Homo sapiens
CQETCCRPSCCETTCCRTTCCRPSCCVSSCCRPQCCQSVCCQPTCSRPSCCQTTCCRTTCYRPSCCVSSCCRPQCCQPVCCQPTCCRPSCCETTCCHPRCCISSCCRPSCCVSSCCKPQCCQSVCCQPNCCRPSCSISSCCRPSCCESSCCRPCCCVRPVCGRVSCHTTCYRPTCVISSCPRPLCCASSCC

SEQ ID NO: 23 - protein, 127 aa, Homo sapiens
MVNSCCGSVCSDQGCDQGLCQETCCRPSCCQTTCCCPSCVVSSCCRPSCSQTTCCQTTCCRPSCCRPVCCQTTCRPSCGVSSCCRPLCCQTTCRPSCGVSSCCRPLCCQTTCCRTTCCRPSCCGSSC

SEQ ID NO: 24 - protein, 201 aa, Homo sapiens
MVNSCCGSVCSDQGCGLENCCRPSCCQTTCCRTTCCRPSCCVSSCCRPQCCQSVCCQPTCCRPSCCQTTCCRTTCCRPSCCVSSCCRPQCCQSVCCQPTCCRPSCCQTTCCRTTCCRPSCCVSSCCRPQCCQSVCCQPTCCRPSCCISSSCCPSCCESSCCRPCCCLRPVCGRVSCHTTCYRPTCVISTCPRPLCCASSCC

SEQ ID NO: 25 - protein, 106 aa, Homo sapiens
MVNSCCGSVCSDQGCGLENCCRPSYCQTTCCRTTCCRPSCCRPSCCRPQCCQSVCCQPTCCCPSYCVSSCCRPQCCQTTRCRTTCCRPSCCVSRCYRPHCGQSLCC

SEQ ID NO: 26 - protein, 195 aa, Homo sapiens
MVNSCCGSVCSHQGCGQDLCQETCCRPSCCETTCCRTTYCRPSCCVSSCCRPQCCQSVCCQPTCCRPRCCISSCCRPSCCVSSCCKPQCCQSMCCQPTCCRPRCCISSCCRPSCCVSSCCRPQCCQSVCCQPTCCHPSCSISSCCRPSCCESSCCRPCCCLRPVCGGVSCHTTCYRPTCVISSCPRPLCCASSCC

SEQ ID NO: 27 - protein, 193 aa, Homo sapiens
QGCGLETCCRPSCCQTTCCRTTCCRPSCCVSSCCRPQCCQSVCCQPTCCRPSCCPSCCQTTCCRTTCCRPSCCVSSCCRPQCCQSVCCQPTCCRPSCSISSCCRPSCCVSRCCRSQCCQSVCCQPTCCRPSCCISSCCRPSCCESSCCRPCCCRPCCCLRPVCGRVSCHTTCYRPTCVISTCPRPLCCASSCC

SEQ ID NO: 28 - protein, 278 aa, Homo sapiens
MGCCGCSGGCGSSCGGCGSGCGGCGSGCGGCGSGCGGSGSSCCVPVCCCKPVCCRVPTCSCSSCGKGGCGSSGGSKGGCGSCGGCKGGCGSCGGSKGGCGSCGGSKGGCGSCGGSKGGCGSGCGGCGSSCCVPVCCCKPMCCCVPACSCSSCGKGGCGSCGCSKGACGSCGGSKGGCGSCGGCKGGCGSCGGSKGGCGSGCGGCGSGCGVPVCCCSCSSCGSCAGSKGGCGSSCSQCSCCKPCCCSSGCGSSCCQSSCCKPCCSQSSCCVPVCCQCKI

SEQ ID NO: 29 - protein, 177 aa, Homo sapiens
MGCCGCSRGCGSGCGGCGSSCGGCGSGCGGCGSGRGGCGSGCGGCSSSCGGCGSRCYVPVCCCKPVCSWVPACSCTSCGSCGGSKGGCGSCGGSKGGCGSCGGSKGGCGSCGCSQSSCCKPCCCSSGCGSSCCQSSCCKPCCCQSSCCVPVCCQSSCCKPCCCQSNCCVPVCCQCKI

SEQ ID NO: 30 - protein, 238 aa, Homo sapiens
MGCSGCSGGCGSSCGGCGSSCGGCGSGYGGCGSGCCVPVCCCKPVCCCVPACSCSSCGSCGGSKGVCGSCGGCKGGCGSCGGSKGGCGSSCCVPVCCSSSCGSCGGSKGVCGFRGGSKGGCGSCGCSQCSCYKPCCCSSGCGSSCCQSSCCKPSCSQSSCCKPCCSQSSCCKPCCCSSGCGSSCCQSSCCKPCCSQSSCCKPCCCSSGCGSSCCQSSCCKPCSSQSSCCVPICCQCKI

SEQ ID NO: 31 - protein, 288 aa, Homo sapiens
MGCCGCSGGCGSGCGGCGSGCGGCGSGCGGCGSGCGGCGSGCGGCGSSCCVPICCCKPVCCCVPACSCSSCGSCGGSKGGYGSCGGSKGGCVSCGGSKGGCGSCGGSKGGCGSCGGSKGGCGSCGGSKGGCVSCGGSKGGCGSCGGSKGGCVSCGGSKGGCGSCGGSKGGCGSCGGSKGGCGSCGGSKGGCGSCGCSQCSCCKPCCCSSGCGSSCCQSSCCKPCCSSSGCGSSCCQSSCCKPYCCQSSCCKPCCSSSGCGSSCCQSSCCNPCCSQSSCCVPVCCQCKI

SEQ ID NO: 32 - protein, 221 aa, Homo sapiens
MGCCGCSGGCGSGCGGLGSGCGGCGSGCGGRGSGCGGCGSGCGGCGSGCGGCGSGCGGCGGCGSGCCVPVCCCKPMCCCVPACSCSSCGKGGCGSCGGSKRGCVSCGVSKGACGSCGGSKGGCGSCGGSKGGCGSCGGSKGGCGSYGCSQCSCCKPCCCSSGCGSSCCQSSCCKPYCCQSSCCKPCSCFSGCGSSCCQSSCYKPCCCQSSCCVPVCCQCKI

SEQ ID NO: 33 - protein, 129 aa, Homo sapiens
MGCCGCSGGCGSGCGGCGSGCGGCGSSCCVPICCCKPVCCCVPACSCTSCGSCGGSKGCCGSCGGSKGGCGSCGGSKGGCGSCGCSQCSCCKPCYCSSGCGSSCCQSSCCKPCCSQASCCVPICCQCKI

SEQ ID NO: 34 - protein, 165 aa, Homo sapiens
MGCCGCSEGCGSGCGGCGSGCGGCGSGCGGCGSSCCVPVCCCKPVCCCVPACSCSSCGSCGGSKGGCGSCGGSKGGCGSCGGSKGGCGSCGCSQCSCYKPCCCSSGCGSSCCQSSCCKPCCCQSSCCKPCCCSSGCGSSCCQSSCCNPCCSQSSCCVPVCCQCKI

SEQ ID NO: 35 - protein, 187 aa, Homo sapiens
MGCCGCSGGCGSGCGGCGSGCGGCGSSCCVPICCCKPVCCCVPACSCSSCGSCGGSKGGRGSCGGSKGDCGSCGGSKGGCGSCGCSQCSCYKPCCCSSGCGSSCCQSSCCKPCCSQSSCCKPCSCSSGCGSSCCQSSCCKPCCSQSSCCKPCCCSSGCGSSCCQSSCCKPCCSQSSCCVPICCQCKI

SEQ ID NO: 36 - protein, 169 aa, Homo sapiens
MGCCGCSGGCGSSCGGCDSSCGSCGSGCRGCGPSCCAPVYCCKPVCCCVPACSCSSCGKRGCGSCGGSKGGCGSCGCSQCSCCKPCCCSSGCGSSCCQCSCCKPYCSQCSCCKPCCSSSGRGSSCCQSSCCKPCCSSSGCGSSCCQSSCCKPCCSQSRCCVPVCYQCKI

SEQ ID NO: 37 - protein, 202 aa, Homo sapiens
MGCCGCSGGCGSGCGGCGSGCGGCGSGCGGYGSGCGGCGSSCCVPVCCCKPVCCCVPACSCSSCGSCGGSKGDCGSCGGSKGGCGSCGGSKGGCGSCGGSKGGCGSCGGSKGGCGSCGGSKGGCGSCGGSKGGCGSCGCSQCNCCKPCCCSSGCGSCCQSSCCNPCCCQSSCCVPVCCQSSCCKPCCCQSSCCVPVCCQCKI

SEQ ID NO: 38 - protein, 156 aa, Homo sapiens
MGCCGCSGGCGSGCGGCGSGSGGCGSGCGGCGSSCCVPICCCKPVCCCVPACSCSSCGSCGGSKGGCGSCGSSKGGCGSCGCSQSNCCKPCCSSSGCGSFCCQSSCSKPCCCQSSCCQSSCCKPCCCQSSCCQSSCFKPCCCQSSCCVPVCCQCKI

SEQ ID NO: 39 - protein, 87 aa, Homo sapiens
MTRYFCCGSYFPGYPIYGTNFHGTFRATPLNCVVPLGSPLNYGCGCNGYSSLGYSFGGSNINNLGGCYGGSFYRPWGSGSGFGYSTY

SEQ ID NO: 40 - protein, 63 aa, Homo sapiens
MLCDNFPGAVFPGCYWGSYGYPLGYSVGCGYGSTYSPVGYGFGYGYNGCGAFGYRRYSPFALY

SEQ ID NO: 41 - protein, 174 aa, Homo sapiens [sequence omitted]

SEQ ID NO: 42 - protein, 159 aa, Homo sapiens
MTHCCSPCCQPTCCRTTCWQPTTVTTCSSTPCCQPSCCVSSCCQPCCHPTCCQNTCCRTTCCQPICVTSCCQPSCCSTPCCQPTCCGSSCGQSSSCAPVYCRRTCYHPTSVCLPGCLNQSCGSNCCQPCCRPACCETTCCRTTCFQPTCVYSCCQPSCC

SEQ ID NO: 43 - protein, 154 aa, Homo sapiens
MTHCCSPCCQPTCCRTTCCRTTCWKPTTVTTCSSTPCCQPSCCVSSCCQPCCRPTCCQNTCCQPTCVTSCCQPSCCSTPCCQPTCCGSSCDQSSSCAPVYCRRTCYYPTTVCLPGCLNQSCGSNCCQPCCRPACCETTCFQPTCVYSCCQPFCC

SEQ ID NO: 44 - protein, 111 aa, Homo sapiens
TTCCQPTCLTSCCQPSCCSTTCCQPICCGSSCCGQTSCGSSCGQSSSCAPVYCRRTCYYPTTVCLPGCLNQSCGSSCCQPCCRPACCETTCCRTTCFQPTCVSSCCQPSCC

SEQ ID NO: 45 - protein, 159 aa, Homo sapiens
MTHCCSPCCQPTCCRTTCWKPTTVTTCSSTPCCQPSCCVSSCCQPCCRPTCCQNTCCQPICVTSCCQPSCCSTPCCQPTCCGQTSCGSSCGQSSSCAPVYCRRTCYHPTTVCLPGCLNQSCGSSCCQPCCRPACCETTCCRTTCFQPTCVYSCCQPSCC

SEQ ID NO: 46 - protein, 154 aa, Homo sapiens
MTHCCSPCCQPTCCRTTCCRTTCWKPTTVTTCSSTPCCQPSCCVSSCCQPCCRPACCQNTCCRTTCCQPTCLSSCCGQTSCGSSCGQSSSCAPVYCRRTCYYPTTVCLPGCLNQSCGSSCCQPCCRPACCETTCCRTTCFQPTCVSSCCQPSCC

SEQ ID NO: 47 - protein, 282 aa, Homo sapiens
MAASTMSVCSSACSDSWQVDACPESCCEPHCCALSCCAPAPCLTLVCTPVSRVSSPCCQAACEPSPCQSGCTSSCTPSCCQQSSCQPACCTSSPCQQACCVPVCCKPVCCLPTCSKDSSSCCQQSSCQPTCCASSSSQQSCCVPVCCKPVCYVPTCSEDSSSCCQQSSCHPACCTSSPCQQACCVPVRCKPVCCKPICCVPVCSGASTSCCQQSSCQPACCTTSCCRPSSSVSLLCRPVCRPACCMPVSSCCAPASSCQASCCRPASCVSLLCRPACSRPAC

SEQ ID NO: 48 - protein, 255 aa, Homo sapiens
MAASTMSICSSACTNSWQVDDCPESCCELPCGTPSCCAPAPCLTLVCTPVSCVSSPCCQAACEPSACQSGCTSSCTPSCCQQSSCQPACCTSSPCQQACCVPVCCKPVCCVPVCCGASSCCQQSSCQPACCASSSCQQSCRVPVCCKAVCCVPTCSESSSSCCQQSSCQPACCTSSPCQQSCCVSVCCKPVCCKSICCVPVCSGASSPCCQQSSCQPACCTSSCCRPSSSVSLLCRPVCSRPASCSFSSGQKSSC

SEQ ID NO: 49 - protein, 221 aa, Homo sapiens
MATSTMSVCSSAYSDSWQVDACPESCCEPPCCATSCCAPAPCLTLVCTPVSCVSSPCCQAACEPSPCQSGCTSSCTPSCCQQSSCQPACCTSSPCQQACCVPVCCKPVCCVPVCCKPVCCKPICCVPVCSGASSSCCQQSSRQPACCTTSCCRPSSSVSLLCRPVCRSTCCVPIPSCCAPASTCQPSCCRPASCVSLLCRPTCSRLSSACCGLSSGQKSSC

SEQ ID NO: 50 - protein, 401 aa, Homo sapiens
MSVCSSDLSYSSRVCLPGSCDSCSDSWQVDDCPESCCEPPCCAPSCCAPAPCLSLVCTPVSRVSSPCCPVTCEPSPCQSGCTSSCTPSCCQQSSCQLACCASSPCQQACCVPVCCKTVCCKPVCCVPVCCGDSSCCQQSSCQSACCTSSPCQQACCVPICCKPVCSGISSSCCQQSSCVSCVSSPCCQAVCEPSPCQSGCISSCTPSCCQQSSCQPACCTSSSCQQACCVPVCCKTVCCKPVCSEDSSSCCQQSSCQPACCTSSPCQQACCVPVCCKPVCCKPVGSVPICSGASSLCCQQSSCQPACCTSSQSQQGCCVPVCCKPVSCVPVCSGASSSCCQQSSCQPACCTTSCCRPSSSVSLLCRPVCRPACCVPVPSCCAPTSSCQPSCCRPASCVSLL

SEQ ID NO: 51 - protein, 271 aa, Homo sapiens
MAACTMSVCSSACSDSWRVDDCPESCCEPPCGTAPCLTLVCTPVSCVSSPCCQAACEPSPCQSGCTSSCTPSCCQPACCASSPCQQACCVPVCCKPVCCLPTCSKDSSSCCQQSSCQPTCCASSSCQQSCCVPVCCKPVCCVPTCSEDSSSCCQHSSCQPTCCTSSPCQQSCYVPVCCKPVCFKPICCVPVCSGASTSCCQQSSCQPACCTTSCCRPSSSVSLLCRPICRPACCVPISSCCAPASSYQASCCRPASCVSLLCRPACSRLAC

SEQ ID NO: 52 - protein, 365 aa, Homo sapiens
MAASTMSVCSSDLSYGSRVCLPGSCDSCSDSWQVDDCPESCCEPPCCAPAPCLSLVCTPVSRVSSPCCPVTCEPSPCQSGCTSSCTPSCCQQSSCQLACCASSPCQQACCVPVCCKTVCCKPVCCVSVCCGDSSCCQQSSCQSACCTSSPCQQACCVPVCCKPVCSGISSSCCQQSSCVSCVSSPCCQAVCEPSPCQSGCTSSCTPSCCQQSSCQPTCCTSSPCQQACCVPVCCVPVCCVPTCSEDSSSCCQQSSCQPACCTSSPCQHACCVPVCSGASTSCCQQSSCQPACCTASCCRPSSSVSLLCHPVCKSTCCVPVPSCGASASSCQPSCCRTASCVSLLCRPMCSRPACYSLCSGQKSSC

SEQ ID NO: 53 - protein, 375 aa, Homo sapiens
MAASTMSVCSSDLSYGSRVCLPGSCDSCSDSWQVDDCPESCCEPPCCAPSCCAPAPCLSLVCTPVSRVSSPCCPVTCEPSPCQSGCTSSCTPSCCQQSSCQLACCASSPCQQACCMPVCCKTVCCKPVYCVPVCSGDSSCCQQSSCQSACCTSSPCQQACCVPICCKPVCSGISSSCCQQSSCVSCVSSPCCQAVCEPSPCQSGCISSCTPSCCQQSSCQPACCTSSPCQQACCVPVCCKPVCCVPTCSDDSGSCCQPACCTSSQSQQGCCVPVCCKPVCCVPVCSGASSSCCQQSSCQPACCTTSCCRPSSSVSLLCRPVCRPTCCVPVPSCCAPTSSCQASCCRPASCVSLLCRPACSRPACCGPTSTQKSSC

SEQ ID NO: 54 - protein, 259 aa, Homo sapiens
MADACCTRTYVIAASTMSVCSSDVGHVSRVSSPSTCTGSSWQVDNCQESCCEPRSCASSCCTPSCCAPAPCLALVCAPVSCEPSPCQSGCTDSCTPSCCQQSSCQPACCTSSPCQQACCVPVCCKSNCCKPVCCVSICSGASSPCCQQSSCQSACCTFSPCQQACCVPICCKPICCVPVCSGASSLCCQKSSCQPACCTTSCCRPSSSVSLLCRPVCRPACCVPVPSCCVPASSCQPSCCHPASCLSFLCRPACSRLAC

SEQ ID NO: 55 - protein, 292 aa, Homo sapiens
MAASTMSIRSSAYSDSWQVDDCPESCCEPPCCATSCCAPAPCLTLVCTPVSRVSSPCCQVTCEPSPCQSGCTSSCTPSCCQQSSCQPAYCTSSPCQQACCVPVCCKPVCCVPVCCGASSCCQQSSYQPACCASSSCQPACCVPVCCKPVCCAPTCSEDSYSCCQQSSCQPACCTSSPCQQSYCVPVCCKPVCCKPICCVPVCSGASSLCCQQSGCQPACCTTSCCRPSSSVSLLCRPVCRPACCVPVSSCCAPTSSRQPSYCRQASCVSLLCRPVCSRPACYSFSSGQKSSC

SEQ ID NO: 56 - protein, 251 aa, Homo sapiens
MAASTMSICSSACTDSWRVVDCPESCCEPCCCAPAPSLTLVCTPVSCVSSPCCQTACEPSACQSGYTSSCTTPCYQQSSCQPDCCTSSPCQQACCVPVCCVPVCCVPVCNKPVCFVPTCSESSPSCCQQSSCQPTCCTSSPCQQACCVPVCSKSVCYVPVCSGASTSCCQQSSCQPACCTASCCRPSSSVSLLCHPVCKSTCCVPVPSCGASASSCQPSCCRTASCVSLLCRPVCSRPACYSLCSGQKSSC

SEQ ID NO: 57 - protein, 298 aa, Homo sapiens
MAASTMSVCSSAYSDSWQVDDCPESCCEPPCSAPSCCAPAPSLSLVCTPVSCVSSPCCQAACEPSACQSGCTSSCTPSCCQQSSCQPACCTSSPCQQACCVPVCCKTVCCKPVCCVPVCCGAASSCCRQSSCQPACCASSSCQPACCVPVCCKPVCCVSTCSEDSSSCCQQSSCQPACCTSSSYQQACCVPVCCKTVYCKPICCVPVCSRASSSRCQQPSCQPACCTTSCCRPSSSVSLLCHPVCRSTCCVPVSSCCAPTSSCQSSCCCPASCVSLLCRPASSRLACYSLCSGKKSSC

SEQ ID NO: 58 - protein, 245 aa, Homo sapiens
MSVCSSDLSYGSRVCLPGSCDSCSDSWQVDDCPESCCEPPCCAPAPCLSLVCTPVSRVSSPCCRVTCEPSPCQSGCTSSCTPSCCQQSSCQPACCTSSPCQQACCVPVCCKTVCCKPVCCMPVCCGPSSSCCQQSSCQPACCISSPCQQSCCVPVCCKPICCVPVCSGASSLCCQQSSCQPACCTTSCCRPSSSVSLLCRPVCRPARRVPVPSCCVPTSSCQPSCGRLASCGSLLCRPTCSRLAC

SEQ ID NO: 59 - protein, 163 aa, Homo sapiens
MSFNCSTRNCSSRPIGGRCIVPVAQVTTTSTTDADCLGGICLPSSFQTGSWLLDHCQETCCEPTACQPTCYRRTSCVSNPCQVTCSRQTTCISNPCSTTYSRPLTFVSSGCQPLGGISSVCQPVGGISTVCQPVGGVSTVCQPACGVSRTYQQSCVSSCRRTC

SEQ ID NO: 60 - protein, 96 aa, Homo sapiens
MCHTSCSSGCQPACCAPSPCQASCYIPVGCQSSVCVPVSFKPAVCVPVRCQSSVCVPVSCRPVVYAAPSCQSSGCCQPSCTSVLCRPISCSTPSCC

SEQ ID NO: 61 - protein, 146 aa, Homo sapiens
MCHTSCSSGCQPACCAPSPCQPACCVPSSCQASCCVPVGCQSSVCVPVSFKPAVCLPVSCQSSVCVPMSFKSAVCVPVSCQSSVCVPVSCRPIVCAAPSCQSSLCVPVSCRPVVYAAPSCQSSGCCQPSCTSVLCRPISYSISSCC

SEQ ID NO: 62 - protein, 96 aa, Homo sapiens
MCHTSCSPACQPTCCIRSPCQASCYVPVSCQSSVCMPVSCTRIVCVAPSCQPSVCVPVSCRPIIYVTPSCQSSGCCQPPCTTALCRPISCSTPSCC

SEQ ID NO: 63 - protein, 112 aa, Homo sapiens
MCHTSHSSGCPMACPGSPCCVPSTCYPPEGYGTSCCCSAPCVALLCRPLCGVSTCCQPACCVPSPCQVACCVPVSCKPVLCVASFCPTSGCCQPFCPTLVYRPVTWSTPTGC

SEQ ID NO: 64 - protein, 172 aa, Homo sapiens
MSYNCCSGNFSSRSCGGYLHYPASSCGFSYPSNQVYSTDLCSPSTCQLGSSLYRGCQQTCWEPTSCQTSYVESSPCQTSCYRPRTSLLCSPCQTTYSGSLGFGSSSCRSLGYGSRSCYSVGCGSSGFRSLGYGGCGFPSLGYGVGFCRPTYLASRSCQSSCYRPTCGSGFYY

SEQ ID NO: 65 - protein, 105 aa, Homo sapiens
MGCCPGDCFTCCTQEQNCCEECCCQPGCCGCCGSCCGCGGSGCGGSGCGGSCCGSSCCGSGCGGCGGCGGCGGGCCGSSCCGSSCCGSGCCGPVCCQPTPICDTK

SEQ ID NO: 66 - protein, 88 aa, Homo sapiens
HYGSYYGGLGYSCGGFGGLGYGYGCGCGSFCRRGSGCGYGGYGYGSGFGSYGYGSGFGGYGYGSGFGGYGYGCCRPSYNGGYGFSGFY

SEQ ID NO: 67 - protein, 11 aa, Homo sapiens
  MISC_FEATURE (2)..(2): xaa = Pro or Xaa is any amino acid
SXCCXPSCCXP

SEQ ID NO: 68 - protein, 10 aa, Homo sapiens
  MISC_FEATURE (4)..(4): xaa = Tyr, Leu or Phe
YGGXGYGSGY

(19) United States
(12) Patent Application Publication
(10) Pub. No.: US 2006/0272103 A1
(43) Pub. Date: Dec. 7, 2006

(54) Compositions comprising KAP polypeptides

(75) Inventor: Philippe Barbarat, Bois-Colombes (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/377,336

(22) Filed: Mar. 17, 2006

Related U.S. Application Data
(60) Provisional application No. 60/666,312, filed on Mar. 30, 2005.

(30) Foreign Application Priority Data
Mar. 18, 2005 (FR) 05 50712

(51) Int. Cl. A61K 8/00

(52) U.S. Cl. 8/405

(57) ABSTRACT

Compositions containing at least one KAP polypeptide and at least one cosmetic ingredient chosen from conditioning agents, care agents and/or treating agents for keratin substrates, and mixtures thereof. The invention also relates to a cosmetic process for caring for, strengthening and/or repairing keratin substrates, in particular keratin fibres, using the compositions.

### REFERENCE TO PRIOR APPLICATIONS

This application claims priority to U.S. provisional application 60/666,312 filed Mar. 30, 2005, and to French patent application 0550712 filed Mar. 18, 2005, both incorporated herein by reference.

### FIELD OF THE INVENTION

The field of the invention relates to the care, strengthening and/or repair of keratin substrates and in particular of keratin fibres.

The invention relates especially to compositions comprising, preferably in a physiologically-acceptable medium suitable for topical application to keratin substrates, at least one polypeptide of the KAP family and at least one cosmetic ingredient chosen from conditioning agents, care agents and/or treating agents for keratin substrates, and mixtures thereof.

The invention also relates to a cosmetic process for caring for, strengthening and/or repairing keratin substrates, in particular keratin fibres, using the compositions.

The invention also relates to the cosmetic use of at least one KAP polypeptide in a composition, as an agent for maintaining and/or restoring and/or improving the protein content of the keratin substrate and thereby conserving and/or restoring and/or improving the physical and/or mechanical properties of keratin substrates.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive..

### BACKGROUND OF THE INVENTION

Keratins are fundamental compounds of the skin, the hair, the eyelashes and the nails. These water-insoluble fibrous proteins contribute especially towards their form, elasticity and strength.

In the hair, for example, there are two major types of protein:
the α keratins, which are divided especially into two groups—the acidic keratins (hHa1-8) and the basic to neutral keratins (hHb1-6)—are long-chain helical polypeptides supercoiled in a helices, which form the intermediate microfibrils or filaments of the cortex (FI), inserted in a matrix of KAPs;
the KAPs (Keratin-Associated Proteins) or IFAPs (Intermediate Filaments Associated Protein) form the amorphous matrix between these microfibrils. There are 3 types of KAP divided among 23 families to date: the highly cysteine-rich KAPs (UHS or Ultra High Sulfur, which contain more than 30 mol % of cysteine), the cysteine-rich KAPs (HS or High Sulfur, which contain less than 30 mol % of cysteine) and the glycine- and tyrosine-rich KAPs (HGT or High Glycine and Tyrosine).

These proteins together provide, by means of covalent bonds (especially disulfide bridges), saline bonds, hydrogen bonds or hydrophobic bonds between the amino acid chains, the cohesion of the three-dimensional structure of the hair. In particular, the sequences of the a keratins and of the KAPs have repeating units, one rich in lysine residues, the other in glutamine residues, which constitute potential substrates for enzymes (e.g.: transglutaminases) capable of forming interfilament bridges (FI-FI), interKAP bridges (KAP-KAP) or covalent bonds between the keratin filaments and the KAPs (FI-KAP), playing an essential role in the structure and shape of the hair shaft.

Keratin substrates and in particular keratin fibres may be sensitized or weakened by the chemical action of atmospheric agents (UV, pollution, etc.) and/or treatments. (bleaching, dyeing, relaxing, permanent-waving, etc.).

It is known, for example, that sensitized or embrittled hair is often dry, coarse and difficult to disentangle and to style.

It is also known that the nails frequently present structural and consistency defects, the effect of which is to make the surface of the nails unattractive, which may be a source of embarrassment and much inconvenience.

L'Oréal patent application WO 99/49837 discloses the use of polyamino acid derivatives for strengthening and caring for keratin fibres.

Patent application WO 03/042387 describes nucleotide and polypeptide sequences of KAP type with hair keratin binding activity, and also cosmetic or therapeutic agents incorporating the-sequences.

### SUMMARY OF THE INVENTION

However, to the inventor's knowledge, no description or suggestion has ever been made of cosmetic compositions comprising at least one KAP polypeptide and at least one ingredient chosen from conditioning agents, care agents and/or treating agents for keratin substrates, and mixtures thereof, the combination being intended especially to improve the appearance and/or the surface state of keratin substrates, and to preserve and/or improve the physical and/or mechanical properties of keratin substrates, in particular when the keratin substrates are sensitized or weakened by the chemical action of atmospheric and/or treating agents.

### DETAILED DESCRIPTION OF THE PREFERRED EMBODIMENTS

The invention thus relates to a composition comprising, preferably in a physiologically acceptable medium suitable for topical application to keratin substrates, at least one polypeptide of the KAP family or peptide fragment thereof (“a KAP polypeptide”) and at least one ingredient chosen from conditioning agents, care agents and/or treating agents for keratin substrates, and mixtures thereof.

The term “keratin substrate” according to the invention covers the skin, the nails and keratin fibres. The term “keratin fibres” means head hair, the eyelashes, the eyebrows and other bodily hair.

According to the invention, the term “physiologically acceptable medium” means a cosmetically acceptable medium that is compatible with keratin substrates.

In general, a medium that is compatible with keratin substrates may be anhydrous or aqueous; it may thus comprise an aqueous phase and/or a fatty phase.

The term “aqueous phase” means water or a mixture of water and of hydrophilic organic solvent(s), for instance alcohols and especially linear or branched lower monoalcohols containing from 2 to 5 carbon atoms, for instance ethanol, isopropanol or n-propanol, and polyols, for instance glycerol, diglycerol, propylene glycol, sorbitol, pentylene glycol and polyethylene glycols, or alternatively hydrophilic C₂ ethers and C₂-C₄ aldehydes. Water or a mixture of water and of hydrophilic organic solvents may be present in the composition according to the invention in a content ranging from 0.1% to 99% by weight and preferably from 10% to 80% by weight relative to the total weight of the composition.

The term “fatty phase” means a phase comprising at least one fatty substance, chosen from oils, waxes and pasty fatty substances, and mixtures thereof.

In particular, the polypeptide of the KAP family that may be used in the compositions of the invention has a peptide sequence chosen from (i) the sequences SEQ ID No. 1 to SEQ ID No. 66, (ii) a fragment of the sequences comprising at least 3 consecutive amino acids of a repeating sequence chosen from the sequence SZCCXPSCCXP (Z: Ø, P and X: Q,V,R,I) and the sequence YGGXGYGSGY (X:Y,L,F) and (iii) homologues thereof. “Z” may correspond to a proline (P) amino acid or any amino acid (Ø).

The sequences SEQ ID No. 1 to SEQ ID No. 66 are given in the appendix, incorporated herein by reference.

The fragments (ii) comprising at least 3. consecutive amino acids of a repeating sequence chosen from the sequence SZCCXPSCCXP (Z: Ø, P and X: Q,V,R,I) and the sequence YGGXGYGSGY (X:Y,L,F) will advantageously be potential substrates of enzymes (e.g.: transglutaminases) capable of forming interfilament bridges (FI-FI), inter-KAP bridges (KAP-KAP) or covalent bonds between keratin filaments and KAPs (FI-KAP).

In particular, the fragment containing at least 3 consecutive amino acids of the repeating sequence SZCCXPSCCXP (Z: Ø, P and X: Q,V,R,I) and the sequence YGGXGYGSGY (X:Y,L,F), preferably of at least 4 amino acids and even more preferentially of at least 5 consecutive amino acids of the repeating sequence, will comprise at least one amino acid capable of forming covalent bonds (e.g.: cysteine), saline bonds (e.g.: lysine, arginine, histidine, aspartate or glutamate), hydrogen bonds (e.g.: serine or tyrosine) or hydrophobic bonds (e.g.: glycine, alanine, valine, leucine or isoleucine), either with the intermediate keratin filaments (FI) or with other KAPs, or alternatively with other constituent proteins of the hair.

The fragment (ii) may preferably contain from 3 to 60 amino acids, in particular from 3 to 20 amino acids and even more preferentially from 3 to 10 amino acids.

This polypeptide fragment may be obtained by proteolysis or synthetically according to the known methods.

According to one particular embodiment, the sequence SEQ ID No. 9 or, preferably, a fragment of this sequence SEQ ID No. 9, in particular the fragment SPCCR, may be used.

According to the invention, the term “homologues” means any peptide sequence that is at least 50%, preferably at least 80% and even more preferentially at least 95% identical to the peptide sequence (i) chosen from the sequences SEQ ID No. 1 to SEQ ID No. 66 or to the fragment (ii) as defined above, in the same species or in a different species: in the latter case, it is also referred to as an “orthologous polypeptide”.

The term “percentage of identity” between two peptide sequences or amino acid sequences is intended to denote a percentage of amino acid residues that are identical between the two sequences to be compared, obtained after the best alignment, i.e. the optimum alignment achieved, for example, using the Smith-Waterman local homology algorithm (1981, Ad. App. Math. 2: 482), using the Neddleman-Wunsch local homology algorithm (1970, J. Mol. Bio. 48: 443), using the Pearson-Lipman similarity search method. (1988, Proc. Natl. Acad. Sci. USA 85:2444), or using computer software using these algorithms (GAP, BESTFIT, BLAST P and BLAST N available on the site NCBI, FASTA and TFASTA in Wisconsin Genetics Software Package, Genetics Computer Group, 575 Science Dr, Madison, Wis.).

The KAP polypeptides according to the invention may be polypeptides of natural or synthetic origin.

The term “natural origin” means a polypeptide in pure form or dissolved at various concentrations, obtained via various extraction processes from a keratin material (skin, nail or hair, in particular hair) of natural origin.

The term “synthetic origin” means a polypeptide in pure form or dissolved at various concentrations, obtained chemically or via production in an organism after introducing into this organism the components required for this production. For example, the polypeptide will be a recombinant polypeptide.

The polypeptide of the KAP family is preferably present in the composition in an amount that is sufficient to obtain the desired effect, i.e. to maintain and/or restore and/or improve the protein content of keratin substrates. In particular, this amount may range from 0.001% to 30% by weight relative to the total weight of the composition, preferably from 0.01% to 15% and even more preferentially from 0.1% to 5% by weight relative to the total weight of the composition.

The term “conditioning agent for keratin substrates” especially includes according to the invention oils, waxes, ceramides, gums, silicones, polysiloxanes, film-forming polymers or fixing polymers, and mixtures thereof.

This agent will especially be intended to improve the appearance and/or the surface state of keratin substrates (e.g.: softer, smoother, less split hair, shinier nails, etc.), to preserve and/or improve the physical and/or mechanical properties of keratin substrates (e.g.: stronger hair that is easier to style, less brittle nails, etc.), in particular when the keratin substrates are sensitized or weakened by the chemical action of atmospheric and/or treating agents.

Examples of oils that may be used include hydrogenated or non-hydrogenated natural oils, saturated or unsaturated, linear or branched, cyclic or aliphatic hydrocarbon-based synthetic oils, for instance poly-α-olefins, in particular polydecenes and polyisobutenes, volatile or non-volatile, organomodified or non-organomodified, soluble or insoluble silicone oils, fluoro or perfluoro oils, fatty esters, esters of polyhydric alcohols and glycerides.

Examples that may be mentioned include hydrocarbon-based oils of animal origin such as perhydrosqualene; hydrocarbon-based plant oils such as liquid triglycerides of fatty acids containing from 4 to 10 carbon atoms, for instance heptanoic or octanoic acid triglycerides, or alternatively sunflower oil, corn oil, soybean oil, grapeseed oil, sesame seed oil, apricot oil, macadamia oil, castor oil, avocado oil, caprylic/capric acid triglycerides, jojoba oil or shea butter; linear or branched hydrocarbons, of mineral or synthetic origin, such as liquid paraffins and derivatives thereof, petroleum jelly, polydecenes, and hydrogenated polyisobutene such as parleam; synthetic esters and ethers, especially of fatty acids, for instance purcellin oil, isopropyl myristate, 2-ethyl-hexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates or decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol or oleyl alcohol; partially hydrocarbon-based and/or partially silicone-based fluoro oils; silicone oils, for instance volatile or non-volatile, linear or cyclic polymethylsildxanes (PDMS) that are liquid or pasty at room temperature, for instance cyclomethicones, dimethicones optionally comprising a phenyl group, for instance phenyl trimethicones, phenyltrimethylsiloxydiphenylsiloxanes, diphenylmethyl dimethyl trisiloxanes, diphenyl dimethicones, phenyl dimethicones and polymethyl phenylsiloxanes; mixtures thereof.

These oils may be present in any amount including in a content ranging from 0.01% to 90% and better still from 0.1% to 85% by weight relative to the total weight of the composition.

It is also possible to use synthetic or natural waxes of plant, mineral or animal origin, with a melting point of greater than or equal to 30° C., which may be up to 120° C. These waxes may be hydrocarbon-based waxes, fluoro waxes and/or silicone waxes. Examples that may be mentioned include beeswax, carnauba wax, candelilla wax, paraffin wax, microcrystalline waxes, ceresin and ozokerite; synthetic waxes, for instance polyethylene wax or Fischer-Tropsch wax, silicone waxes, for instance alkyl or alkoxy dimethicones containing from 16 to 45 carbon atoms, and ceramides.

Silicone gums and resins, quaternized or non-quaternized proteins or protein hydrolysates, or a mixture of these various agents, may also be used. Mention may also be made of volatile or non-volatile, linear or cyclic silicones and mixtures thereof, polydimethylsiloxanes, quaternized polyorganosiloxanes, polyorganosiloxanes containing aminoalkyl groups modified with alkoxycarbonylalkyl groups, polyorganosiloxanes such as polydimethylsiloxane-polyoxyalkyl copolymer of the dimethicone copolyol type, a polydimethylsiloxane containing stearoxy end groups (stearoxydimethicone), a polydimethylsiloxane-dialkylammonium acetate copolymer or a polydimethylsiloxanepolyalkylbetaine copolymer, pplysiloxanes organo-modified with mercapto or mercaptoalkyl groups such as those described in French patent FR 1 530 369 and in European patent application EP 295 780, and also silanes such as stearoxytrimethylsilane.

Film-forming polymers that may be mentioned include synthetic polymers, of radical type or of polycondensate type, polymers of natural origin, and mixtures thereof. In particular, mention may be made of acrylic polymers, polyurethanes, polyesters, polyamides, polyureas and cellulose-based polymers, for instance nitrocellulose. In aqueous medium, polyurethanes, for example anionic polyurethanes, polyester-polyurethanes, polyether-polyurethanes, radical polymers, especially of acrylic, styrene-acrylic and/or vinyl type, polyesters and alkyd resins will preferentially be used, alone or as a mixture.

As fixing polymers, which may be used especially in compositions for shaping and/or styling and/or holding the hair, mention may be made of anionic fixing polymers, cationic fixing polymers, nonionic fixing polymers and amphoteric fixing polymers, and mixtures thereof.

Examples of anionic fixing polymers that may be mentioned include those containing carboxylic groups, such as homopolymers or copolymers of acrylic or methacrylic acid or salts thereof, copolymers of acrylic or methacrylic acid with a monoethylenic monomer such as ethylene or styrene, vinyl esters, acrylic or methacrylic acid esters, optionally grafted onto a polyalkylene glycol such as polyethylene glycol, and optionally crosslinked, crotonic acid copolymers, copolymers of C₄-C₈ monounsaturated carboxylic acids or anhydrides, and polyacrylamides comprising carboxylate groups.

Examples of cationic fixing polymers that may be mentioned include copolymers of acrylamide and of dimethylaminoethyl methacrylate quaternized with dimethyl sulfate or with a dimethyl halide; cationic polysaccharides, preferably containing a quaternary ammonium, such as guar gums containing cationic trialkylammonium groups; quaternary copolymers of vinylpyrrolidone and of vinylimidazole; chitosans or salts thereof; the salts that may be used are in particular chitosan acetate, lactate, glutamate, gluconate or pyrrolidonecarboxylate; cationic cellulose derivatives such as cellulose copolymers or cellulose derivative copolymers grafted with a water-soluble monomer comprising a quaternary ammonium.

Amphoteric fixing polymers that may be mentioned include copolymers containing acidic vinyl units and containing basic vinyl units.

The term “agent for caring for and/or treating keratin materials” preferably means lipophilic or hydrophilic active agents used in compositions for, caring for and/or treating the skin, the hair, the eyelashes and the nails.

These agents will especially be intended to improve the appearance and/or surface state of keratin substrates, and to preserve and/or improve the physical and/or mechanical properties of keratin substrates, in particular when the keratin substrates are sensitized or weakened by the chemical action of atmospheric and/or treating agents.

For caring for and/or treating the skin, mention may be made especially of desquamating or moisturizing agents, depigmenting or pro-pigmenting agents, agents for stimulating the synthesis of dermal or epidermal macromolecules and/or for preventing their degradation, anti-glycation agents, NO-synthase inhibitors, anti-pollution agents or free-radical scavengers, calmatives, agents that act on the capillary circulation, etc.

Desquamating Agents

The term “desquamating agent” means any compound capable of acting:

either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives;

or on the enzymes involved in the desquamation or degradation of corneodesmosomes, glycosidases, stratum corneum chymotryptic enzyme (SCCE), or even other proteases (trypsin, chymotrypsin-like?). Mention may be made of agents for chelating mineral salts: EDTA; N-acyl-N,N′,N′-ethylenediaminetriacetic acid; amino-sulfonic compounds and in particular (N-2-hydroxyethyl-piperazine-N-2-ethane)sulfonic acid (HEPES); 2-oxo-thiazolidine-4-carboxylic acid (procysteine) derivatives; α-amino acid derivatives of the glycine type (such as described in EP-0 852 949 and sodium methylglycinediacetate sold by BASF under the trade name Trilon M); honey; sugar derivatives such as O-octanoyl-6-D-maltose and N-acetylglucosamine.

Moisturizers

The term “moisturizer” means:

either a compound acting on the barrier function, in order to keep the stratum corneum moisturized, or an occlusive compound. Mention may be made of ceramides, sphingoid-based compounds, lecithins, glycosphingo-lipids, phospholipids, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;

or a compound that directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides such as the product sold under the reference Pentavitin, honey, alginates (in particular the product Sobalg PH 154 sold by Grindsted), cyclic carbonates, N-lauroylpyrrolidonecarboxylic acid or salts thereof, in particular the sodium salt sold under the reference Nalidone, and N-α-benzoyl-L-arginine;

or a compound that activates the sebaceous glands, such as steroid derivatives (including DHEA, the 7-oxido and/or 17-alkyl derivatives thereof, and sapogenins), methyl dihydrojasmonate and vitamin D and its derivatives.

These compounds may represent any amount of the compositon, such as from 0.001% to 30% and preferably from 0.01% to 20% of the total weight of the composition according to the invention.

Depigmenting, Anti-Pigmenting or Pro-Pigmenting Agents

The depigmenting or anti-pigmenting agents that may be incorporated into the composition according to the present invention comprise, for example, the following compounds: kojic acid; ellagic acid; arbutin and its derivatives such as those described in patent applications EP-895 779 and EP-524 109; hydroquinone; aminophenol derivatives such as those described in patent applications WO 99/10318 and WO 99/32077, and in particular N-cholesteryloxycarbonyl-para-aminophenol and N-ethyloxycarbonyl-para-aminophenol; iminophenol derivatives, in particular those described in patent application WO 99/22707; L-2-oxothiazolidine-4-carboxylic acid or procysteine, and also its salts and esters; calcium D-pantheteine sulfonate; ascorbic acid and its derivatives, especially ascorbyl glucoside; and plant extracts, in particular extracts of liquorice, of mulberry, of skullcap and of Bacopa monnieri, without this list being limiting.

Pro-pigmenting agents that-may be mentioned include the extract of burnet (Sanguisorba officinalis) sold by the company Maruzen, and extracts of chrysanthemum (Chrysanthemum morifolium).

Agents for Stimulating the Synthesis of Dermal or Epidermal Macromolecules and/or for Preventing their Degradation

Among the active agents for stimulating dermal macromolecules or for preventing their degradation, particular mention may be made of those that act:

either on collagen synthesis, such as extracts of Centella asiatica; asiaticosides and derivatives; ascorbic acid or vitamin C and its derivatives; synthetic peptides such as iamin, biopeptide CL or the palmitoyloligopeptide sold by the company Sederma; peptides extracted from plants, such as, the soybean hydrolysate sold by the company Coletica under the trade name Phytokine®; and plant hormones such as auxins and lignans;

or on elastin synthesis, such as the extract of Saccharomyces cerivisiae sold by the company LSN under the trade name Cytovitin®; and the extract of the alga Macrocystis pyrifera sold by the company SECMA under the trade name Kelpadelie®;

or on glycosaminoglycan synthesis, such as the product of fermentation of milk with Lactobacillus vulgaris, sold by the company Brooks under the trade name Biomin yogourth®; the extract of the brown alga Padina pavonica sold by the company Alban Müller under the trade name HSP3®; and the extract of Saccharomyces cerevisiae available especially from the company Silab under the trade name Firmalift® or from the company LSN under the trade name Cytovitin®;

or on fibronectin synthesis, such as the extract of the zooplankton Salina sold by the company Seporga under the trade name GP4G®;

the yeast extract available especially from the company Alban Müller under the trade name Drieline®; and the palmitoyl pentapeptide sold by the company Sederma under the trade name Matrixil®;

or on the inhibition of metalloproteases (MMP), such as, more particularly, MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon, or of sage;

or on the inhibition of serine proteases such as *leukocyte elastase or cathepsin G. Mention may be made of: the peptide extract of Leguminosa seeds (Pisum sativum) sold by the company LSN under the trade name Parelastyl®; and heparinoids and pseudodipeptides such as {2-[acetyl(3-trifluoromethylphenyl)amino]-3-methyl-butyrylamino}acetic acid.

Among the active agents that stimulate epidermal macromolecules, such as fillagrin and keratins, mention may be made especially of the extract of lupin sold by the company Silab under the trade name Structurine®; the extract of beech Fagus sylvatica buds sold by the company Gattefosse under the trade name Gatuline®; and the extract of the zooplankton Salina sold by the company Seporga under the trade name GP4G®.

Anti-Glycation Agents

The term “anti-glycation agent” means a compound for preventing and/or reducing the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetrahydroxystilbene. These anti-glycation agents are described in patent applications FR 2 802 425, FR 2 810 548, FR 2 796 278 and FR 2 802 420, respectively. Resveratrol is particularly preferred for use in this invention.

NO-Synthase Inhibitors

Examples of NO-synthase inhibitors that are suitable for use in the present invention especially comprise a plant extract of the species Vitis vinifera which is sold especially by the company Euromed under the name “Leucocyanidines de raisins extra”, or by the company Indena under the name Leucoselect®, or finally by the company Hansen under the name “Extrait de marc de raisin”; a plant extract of the species Olea europaea which is preferably obtained from olive tree leaves and is sold especially by the company Vinyals in the form of a dry extract, or by the company Biologia & Technologia under the trade name Eurol BT; and a plant extract of the species Gingko biloba which is preferably a dry aqueous extract of this plant sold by the company Beaufour under the trade name “Gingko biloba extrait standard”.

Anti-Pollution Agents or Free-Radical Scavengers

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be used in the composition according to the invention, mention may be made in particular of vitamin C and its derivatives including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolysed RNA, sold by Laboratoires Sérobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C-49® by the company Induchem, and the mixture of extracts of ginseng, of apple, of peach, of wheat and of barley, sold by the company Provital under the trade name Pronalen Bioprotect®.

As agents for trapping monocyclic or polycyclic aromatic compounds, which may be used in the composition according to the invention, mention may be made in particular of tannins such as ellagic acid; indole derivatives, in particular 3-indolecarbinol; extracts of tea, in particular of green tea, extracts of water hyacinth or Eichhornia crassipes; and the water-soluble fraction of corn sold by the company Solabia under the trade name Phytovityl®.

Finally, as heavy-metal-trapping agents that may be: used in the composition according to the invention, mention may be made in particular of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichhornia crassipes); and the water-soluble fraction of corn sold by the company Solabia under the trade name Phytovityl®.

The free-radical scavengers that may be used in the composition according to the invention comprise, besides certain anti-pollution agents mentioned above, vitamin E and its derivatives such as tocopheryl acetate; bioflavonoids; coenzyme Q10 or ubiquinone; certain enzymes, for instance catalase, superoxide dismutase and extracts of wheatgerm containing it, lactoperoxidase, glutathione peroxidase and quinone reductases; glutathione; benzylidenecamphor; benzylcyclanones; substituted naphthalenones; pidolates; phytanetriol; gamma-oryzanol; quanosine; lignans; and melatonin.

Calmatives

As calmatives that may be used in the composition according to the invention, mention may be made of: pentacyclic triterpenes and extracts of plants (e.g.: Glycyrrhiza glabra) containing them, for instance β-glycyrrhetinic acid and salts and/or derivatives thereof (glycyrrhetinic acid monoglucoronide, stearyl glycyrrhetinate or 3-stearoyloxyglycyrrhetic acid), ursolic acid and its salts, oleanolic acid and its salts, betulinic acid and its salts, extracts of plants such as Paeonia suffruticosa and/or lactiflora, Laminaria saccharina, Boswella serrata, Centipeda cunnighami, Helianthus annuus, Linum usitatissimum, Cola nitida, Epilobium Angustifollum, Aloe vera, Bacopa monieri, salicylic acid salts and in particular zinc salicylate, canola oil, bisabolol and camomile extracts, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, tocotrienols, piperonal, an extract of clove., phytosterols, cortisone, hydrocortisone, indomethacin and betamethasone.

Agents Acting on the Capillary Circulation

The active agents acting on the capillary circulation (vasoprotective or vasodilating agents) may be chosen from flavonoids, ruscogenins, esculosides, escin extracted from common horse chestnut, nicotinates, heperidine methyl chalcone, essential oils of lavender or of rosemary, and extracts of Ammi visnaga.

For caring for and/or treating the hair, examples that may be mentioned include moisturizers, softeners, free-radical scavengers, vitamins-, amino acids, proteins, antidandruff agents, anti-seborrhoeic agents, reducing agents (for shaping or permanently reshaping the hair), hair dye precursors, direct hair dyes, oxidizing agents, etc.

Antidandruff Agents

The antidandruff agents may be any active agent that is useful for preventing the appearance of dandruff, reducing its amount and/or eradicating it entirely. Thus, the antidandruff agents may be chosen from antifungal and/or antibacterial agents.

The antidandruff agents that may )be used according to the invention are especially chosen from:

1) pyridinethione salts, especially the calcium, magnesium, barium, strontium, zinc, cadmium, tin and zirconium salts. The zinc salt of pyridinethione is especially sold under the name zinc Omadine by the company Olin;

2) 1-hydroxy-2-pyrrolidone derivatives, for instance 1-hydroxy-4-methyl-2-pyridone, 1-hydroxy-6-methyl-2-pyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methylcyclohexyl)-2-pyridone, 1-hydroxy-4-methyl-6-(2-bicyclo[2.2.1]heptyl)-2-pyridone, 1-hydroxy-4-methyl-6-(4-methylphenyl)-2-pyridone, 1-hydroxy-4-methyl-6-[1-(4-nitrophenoxy)butyl]-2-pyridone, 1-hydroxy-4-methyl-6-(4-cyanophenoxymethyl)-2-pyridone, 1-hydroxy-4-methyl-6-(phenylsulfonyl-methyl)-2-pyridone and 1-hydroxy-4-methyl-6-(4-bromobenzyl)-2-pyridone. These compounds may be used in the form of salts with organic or mineral bases. A preferred compound is Octopirox (1-hydroxy-4-methyl-6-(2,4,4-trimethylpentyl)-2-pyridone, monoethanolamine salt) sold by the company Hoechst;

3) 2,2′-dithiobis(pyridine N-oxide) in the form- of mineral salts, including the magnesium sulfate;

4) trihalocarbamides;

5) triclosan;

6) azole compounds such as climbazole, ketoconazole, clotrinazole, econazole, isoconazole and miconazole;

7) antifungal polymers such as amphotericin B or nystatin;

8) selenium sulfide;

9) other antidandruff agents are sulfur in its various forms, cadmium sulfide, allantoin, coal tars or wood tars and derivatives thereof, in particular cade oil, salicylic acid, undecylenic acid, fumaric acid, and allylamines such as terbinafine.

Anti-Seborrhoeic Agents

When the composition according to the invention comprises an anti-seborrhoeic agent such as a 5α-reductase inhibitor, this agent may be chosen especially from:
retinoids, and in particular retinol;
sulfur and sulfur derivatives;
zinc salts such as zinc lactate, gluconate, pidolate, carboxylate, salicylate and/or cysteate;
selenium chloride;
vitamin B6 or pyridoxine;
mixture of capryloyl glycine, sarcosine and extract of Cinnamomum zeylanicum sold especially by the company SEPPIC under the trade name Sepicontrol A5®;
an extract of Laminaria saccharina sold especially by the company SECMA under the trade name Phlorogine®;
an extract of Spiraea ulmaria sold especially by the company Silab under the trade name Sebonormine®;
plant extracts from the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;
an extract of Serenoa repens sold especially by the company Euromed;
plant extracts of the genus Silybum; and
extracts of Eugenia caryophyllata containing eugenol and eugenyl glucoside.

Reducing Agents

These agents, which may be used especially in compositions for shaping or permanently reshaping the hair, may be chosen, for example, from thioglycolic acid, glyceryl or glycol. monothioglycolate, cysteamine and C1-C4 acyl derivatives thereof such as N-acetylcysteamine or N-propionylcysteamine, cysteine, N-acetylcysteine, sugar N-mercaptoalkylamides such as N-(2-mercaptoethyl)gluconamide, β-mercaptopropionic acid and derivatives thereof, thiolactic acid and esters thereof such as glyceryl monothiolactate, thiomalic acid, pantheteine, thioglycerol, alkali metal or alkaline-earth metal sulfites or bisulfites, the N-(mercaptoalkyl)hydroxyalkylamides described in patent application EP 354 835 and the N-mono- or N,N-dialkylmercapto-4-butyramides described in patent application EP 368 763, the aminomercaptoalkylamides described in patent application EP 403 267 and the alkylaminomercaptoalkylamides described in patent application EP 432 000.

When the compositions according to the invention contain at least one reducing agent, it is advantageously present in a maximum concentration of 20% by weight and preferably between 0.1% and 10% by weight relative to the total weight of the composition.

Hair Dye Precursors

Mention may be made especially of oxidation dyes, which are generally chosen from oxidation bases, orthodiphenols and couplers, and mixtures thereof.

The oxidation bases, of para or ortho type, are compounds that are not themselves dyes, but form a dye via a process of oxidative condensation, either with themselves or in the presence of a coupler or modifier. They comprise functional groups, either two amino groups, or an amino group and a hydroxyl group in the para or ortho position relative to each other. They may be chosen especially from ortho- and para-phenylenediamines, double bases, ortho- and para-aminophenols, heterocyclic bases, and also the addition salts of all these compounds with an acid.

Ortho-diphenols are compounds comprising at least one aromatic ring in which at least two consecutive carbons bear a hydroxyl group. Preferably, the aromatic ring is a benzene ring or a fused aromatic ring.

The aromatic ring may be a fused aromatic ring optionally containing one or more hetero atoms, such as naphthalene, tetrahydronaphthalene, indane, indene, anthracene, phenanthrene, indole, isoindole, indoline, isoindoline, benzofuran, dihydrobenzofuran, chroman, isochroman, chromene, isochromene, quinoline, tetrahydroquinoline and isoquinoline.

Another class of oxidation dyes consists of couplers, as explained previously. The couplers may be chosen from those conventionally used in oxidation dyeing and especially from meta-aminophenols, meta-phenylene-diamines, meta-diphenols, naphthols and heterocyclic couplers, for instance indole derivatives, indoline derivatives, sesamol and derivatives thereof, pyridine derivatives, pyrazolotriazole derivatives, pyrazolones, indazoles, benzimidazoles, benzothiazoles, benzoxazoles, 1,3-benzodioxoles, quinolines and the addition salts thereof with an acid, these compounds being other than the ortho-dihydroxylated compounds of the invention.

These couplers are more particularly chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diamino-benzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methyl-indole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c)-1,2,4-triazole and 2,6-dimethylpyrazolo[1,5-b)-1,2,4-triazole, and the addition salts thereof with an acid.

Direct Dyes

Among these dyes, which are used especially to modify the shades by enriching them with tints, mention may be made of neutral, cationic or anionic nitro, azo or anthraquinone dyes in a weight proportion of about from 0.001% to 20% and preferably from 0.01% to 10% by weight relative to the total weight of the composition.

Oxidizing Agents

Among the oxidizing agents conventionally used, mention may be made of hydrogen peroxide or compounds capable of producing hydrogen peroxide by hydrolysis, such as urea peroxide or persalts, for instance perborates, percarbonates and persulfates, hydrogen peroxide and persulfates being particularly preferred.

The oxidizing agent is preferably chosen from the group formed by aqueous hydrogen peroxide solution, urea peroxide and persalts such as perborates or persulfates, or mixtures thereof.

For caring for and/or treating the eyelashes, mention may be made especially of agents that promote curling of the eyelashes, such as film-forming polymers (e.g.: acrylic polymers, polyurethanes, polyesters, polyamides, polyureas or cellulose-based polymers, for instance nitrocellulose).

For caring for and/or treating the nails, mention may be made especially of agents that promote nail growth, nail hardeners, etc.

Mention may be made especially of an extract of non-synthetic filamentous bacteria as defined in patent application EP 1 479 367 (L'Oréal). In particular, it will be an extract of Vitreoscilla filiformis.

A person skilled in the art will adapt the amount of cosmetic ingredient in the composition as a function of the desired effect and such that the amount of cosmetic ingredient present in the composition does not affect the desired properties of the KAP polypeptide.

In particular, the cosmetic ingredient(s) will preferably be present in the composition in an amount that can range from 0.001% to 20% by weight relative to the total weight of the composition, more preferably from 0.01% to 10% and even more preferentially from 0.1% to 2% by weight relative to the total weight of the composition.

The composition may also comprise cosmetic adjuvants and/or formulating agents conventionally used in cosmetic compositions, such as organic solvents, surfactants, plasticizers, thickeners, emulsifiers, preserving agents, UV-screening agents, fillers, dyestuffs, fragrances, opacifiers, humectants, agents for adjusting and fixing the pH, antibacterial agents, antifungal agents and propellants, and mixtures thereof.

Organic solvents that may be mentioned include hydrophilic organic solvents, lipophilic organic solvents and amphiphilic solvents, or mixtures thereof.

Examples of hydrophilic organic solvents that may be mentioned include linear or branched lower monoalcohols containing from 1 to 8 carbon atoms, for instance ethanol, propanol, butanol, isopropanol or isobutanol, optionally oxyethylenated polyethylene glycols, polyols such as propylene glycol, isoprene glycol, butylene glycol, glycerol, sorbitol and derivatives thereof, glycol ethers and propylene glycol ethers. Amphiphilic organic solvents that may be mentioned include polyols such as propylene glycol derivatives. Examples of lipophilic organic solvents that may be mentioned include fatty esters.

Mention may also be made of ketones that are liquid at room temperature, such as methyl ethyl ketone, methyl isobutyl ketone, diisobutyl ketone, isophorone, cyclohexanone or acetone; propylene glycol ethers that are liquid at room temperature; short-chain esters (containing from 3 to 8 carbon atoms in total); ethers that are liquid at room-temperature; alkanes that are liquid at room temperature; cyclic aromatic compounds that are liquid at room temperature; aldehydes that are liquid at room temperature, and mixtures thereof.

These solvents may generally be present in a content ranging from 0 to 90% by weight relative to the total weight of the composition, preferably from 0.1% to 90%, more preferably from 10% to 90% and better still from 30% to 90% by weight relative to the total weight of the composition.

Surfactants that may be mentioned include surfactants of nonionic, anionic, cationic or amphoteric type. In particular for hair compositions, mention may be made of alkyl sulfates, alkylbenzene sulfates, alkyl ether sulfates, alkylsulfonates, quaternary ammonium salts, alkylbetaines, oxyethylenated alkylphenols, fatty acid alkanolamides, oxyethylenated fatty acid esters and also other nonionic surfactants of the hydroxypropyl ether type.

Plasticizers that may be mentioned include glycols and derivatives thereof, glycol esters, propylene glycol derivatives, carboxylic acid esters and oxyethylenated derivatives, and mixtures thereof. The plasticizer content may range, for example, from 0.1% to 15% by weight and preferably from 0.5% to 10% by weight relative to the total weight of the composition.

Thickeners that may be mentioned include cellulose and derivatives thereof, for instance cellulose ethers, heterobiopolysaccharides such as xanthan gum, scleroglucans, and crosslinked or non-crosslinked polyacrylic acids. Mention may be made, for example, of natural polymers such as xanthan gum and guar gum or alternatively cellulose derivatives, starches and alginates; synthetic crosslinked acrylic polymers such as the Carbopols® sold by the company Goodrich and crosslinked and at least partially neutralized 2-acrylamido-2-methylpropanesulfonic acid (AMPS) polymers, for instance the product sold under the name Hostacerin® AMPS by the company Clariant.

The thickeners are preferably present in proportions ranging between 0.1% and 5% by weight approximately relative to the total weight of the composition.

Emulsifiers that may be mentioned, inter alia, include lauryl sulfate, triethanolamine stearate or fatty alcohols such as stearyl alcohol or cetyl alcohol.

Among the emulsifiers that may preferably be used in the compositions in the form of a water-in-oil emulsion, mention may be made, inter alia, of glycerol esters, ethoxylated alcohols, lanolin, lanolin alcohol, cholesterol and various sorbitan oleates. The emulsifiers are generally present in a proportion of between 1% and 10% by weight, relative to the total weight of the composition.

Among the emulsifiers for compositions in the form of gels, mention may preferably be made especially of polyoxyethylated ether and oleyl alcohol or the phosphonic ester thereof, polyethoxylated lauryl alcohol, polyethoxylated oleyl alcohol, oxyethylated cetyl alcohol and various polyoxyethylene glycols of fatty acids.

Dyestuffs that may be mentioned include water-soluble dyes and pulverulent dye substrates, for instance the. pigments, nacres and flakes well known to those skilled in the art. The dyestuffs may be present in the composition in a content ranging from 0.01% to 50% by weight and preferably from 0.01% to 30% by weight relative to the weight of the composition.

The term “pigments” should be understood as meaning white or coloured, mineral or organic particles of any form, which are insoluble in the physiological medium, and which are intended to colour the composition.

The pigments may be white or coloured, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D & C type, and lakes based on cochineal carmine or on barium, strontium, calcium or aluminium.

The term “nacres” should be understood as meaning iridescent particles of any form, produced especially by certain molluscs in their shell, or else synthesized.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type, and also nacreous pigments based on bismuth oxychloride.

The water-soluble dyes are, for example, beetroot juice or methylene blue.

The flakes may be chosen from those made of acrylic resin, polyester, polyethylene terephthalate or aluminium.

Fillers that may especially be mentioned include mineral or organic fillers of any form, which are platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate. They are generally present in a content ranging from 0.01% to 50% by weight and preferably ranging from 0.01% to.30% by weight relative to the total weight of the composition.

UV-screening agents or photoprotective agents that may be mentioned include organic UV-screening agents or mineral UV-screening agents (zinc oxide or titanium oxide).

The organic photoprotective agents are chosen especially from anthranilates; cinnamic derivatives; dibenzoylmethane derivatives; salicylic derivatives; camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243, EP 944 624; benzophenone derivatives; β,β-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylene-bis(hydroxyphenylbenzotriazole) derivatives as described in patent applications U.S. Pat. No. 5,237,071, U.S. Pat. No. 5,166,355, GB 2 303 549, DE 197 26 184 and EP 893 119; screening polymers and screening silicones such as those described especially in patent application WO 93/04665; dimers derived from α-alkylstyrene, such as those described in patent application DE 198 55 649; 4,4-diarylbutadienes as described in patent applications EP 0 967 200, DE 197 46 654, DE 197 55 649, EP-A-1 008 586, EP 1 133 980 and EP 133 981, and mixtures thereof.

The mineral photoprotective agents are chosen from pigments or even nanopigments (mean size of the primary particles: generally between 5; nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminium stearate. Such coated or uncoated metal oxide nanopigments are described in particular in patent applications EP 518 772 and EP 518 773.

The screening agents are generally present in the compositions according to the invention in proportions ranging from 0.1% to 20% by weight relative to the total weight of the composition, and preferably ranging from 0.2% to 15% by weight relative to the total weight of the composition.

Preserving agents that may be mentioned include para-hydroxybenzoic acid esters, 1,2-octanediol, 3-iodo-2-propynylbutyl carbamate, phenoxyethanol and chlorhexidine gluconate.

As propellants preferably used in hair mousse or lacquer compositions, examples that may be mentioned include compressed air, carbon dioxide, dinitrogen monoxide, nitrogen, nitrous oxide, dimethyl ether, liquefiable aliphatic hydrocarbons such as propane, butanes including isobutane, pentane, isopentane and neopentane, and mixtures thereof, and chlorinated and/or fluorinated hydrocarbons, or mixtures thereof. Among the chloro and/or fluorohydrocarbons, mention may be made of monochlorodifluoromethane, dichlorodifluoromethane, monochlorodifluoroethane, 1,1-difluoroethane and dichlorotetrafluoroethane, and also mixtures thereof and in particular the mixture of monochlorodifluoromethane/monochlorodifluoroethane (40/60) and the mixture of dichlorodifluoromethane/dichlorotetrafluoroethane (60/40).

The proportion of propellant used is not critical, but it determines the density of the mousse produced. The proportion of propellant is generally between 1% and 20% and preferably between 5% and 15% by weight relative to the total weight of the aerosol composition.

The compositions according to the invention may be in any form, including any galenical form known to be suitable for topical application to keratin substrates (skin, eyelashes, nails or hair).

A person skilled in the art may choose the appropriate form, and also the method for preparing it, on the basis of his general knowledge, taking into account firstly the nature of the constituents used, especially their solubility in the support, and secondly the intended use of the composition.

The compositions according to the invention may especially be in the form of aqueous, aqueous-alcoholic or oily solutions, oil-in-water (O/W), water-in-oil (W/O) or multiple (W/O/W or O/W/O) emulsions, aqueous or oily gels, dehydrated anhydrous products, or dispersions of an oily phase in an aqueous phase using spherules, these spherules possibly being polymer. nanoparticles such as nanospheres and nanocapsules, or lipid vesicles of ionic type (liposomes) and/or nonionic type.

When the composition used according to the invention is an emulsion, the proportion of the fatty phase may range for example from 5% to 80% by weight and preferably from 5% to 50% by weight relative to the total weight of the composition. The oils, emulsifiers and coemulsifiers used in the composition in emulsion form are chosen from those conventionally used in the field under consideration. The emulsifier and the coemulsifier are present in the composition in a proportion ranging from 0.3% to 30% by weight and preferably from 0.5% to 20% by weight relative to the total weight of the composition.

For application in particular to the hair, the composition may preferably be in the form of creams, lotions, gels, emulsions or mousses or in the form of aerosol compositions also comprising a pressurized propellant. It may be in the form of a haircare lotion, for example for daily or twice-weekly application, a shampoo or hair conditioner, in particular for twice-weekly or weekly application, a liquid or solid scalp cleansing soap for daily application, a hairstyle shaping product (lacquer, hairsetting product, styling gel or mousse), a colouring shampoo, a permanent-waving composition, a treating mask, or a foaming cream or gel for cleansing the hair. It may also be in the form of a hair dye or hair mascara to be applied with a brush or a comb.

For application to the eyelashes, the composition will preferably be in the form of a mascara that may be a composition for making up the eyelashes, a base for making up the eyelashes, a composition to be applied over a mascara, also known as a top coat, or a cosmetic treatment composition for the eyelashes. The mascara is more particularly intended for human eyelashes, but also false eyelashes.

For application to the, nails, the composition may preferably be a nail makeup product such as a coloured nail varnish, a nail base or “base coat”, a finishing composition or “top coat”, a composition to be applied under or over the nail makeup product, a nail varnish dissolver or a cosmetic nailcare product such as a treating base for protecting, strengthening and/or repairing the nails.

For application to the skin, the composition may preferably be more or less fluid and may have the appearance of a white or coloured cream, an ointment, a milk, a lotion, a serum, a paste or a mousse. It may also be in solid form, in particular in the form of a stick. It may be used as a care product and/or as a makeup product for the skin.

The compositions can be applied by any suitable means, such as a fine brush, a coarse brush, a spray or with the fingers, for example.

The invention also relates to a cosmetic process for caring for, treating, strengthening and/or repairing keratin substrates, wherein a composition as defined above is applied to the skin, the hair, the eyelashes or the nails, optionally followed by rinsing.

According to the invention, the term “care of keratin substrates” means a composition intended to improve the appearance and/or the surface state of keratin substrates. The care of keratin substrates may consist in making the hair softer, smoother and less split; and in making the nails less brittle and less split.

According to the invention, the term “strengthening and/or repairing keratin substrates” means a composition intended to conserve and/or restore and/or improve the physical and/or mechanical properties of keratin substrates, which may be manifested, for example:

either by rigidification of the keratin substrates, which gives them greater consistency and body, and also a more pleasant feel. This results, for example, in an increase in the volume of the keratin fibres and also greater ease of styling and styling hold;

or by better elasticity and/or better resistance to mechanical tensile forces applied thereto, for example during combing for keratin fibres, in particular on African hair or any embrittled or sensitized hair.

The hair is thus stronger, more supple, softer, smoother and easier to style. The nails are thus smoother, shinier, less damaged, less split and less brittle.

According to a first embodiment, the composition may be applied as a pre-treatment to a treatment liable to sensitize keratin substrates (e.g.: before oxidation dyeing on the hair).

According to another embodiment, the composition may be applied as a post-treatment to a treatment liable to sensitize keratin substrates (e.g.: after a peeling treatment on the skin).

The composition containing the polypeptide of the KAP family may also constitute the treating composition (e.g.: combined with a dye).

The application of the compositions according to the process of the invention may be performed according to the usual technique for using the compositions. For example:
the application to the hair may be performed before (lotion, 1 hour before), during (shampoo) or after (spray lotions) shampooing; the composition may be applied to dry hair (lacquers, sprays or lotions) or to wet hair (permanent-reshaping or hairsetting compositions);
the application of a care base to the nails may be performed before or after applying varnish;
the application of a care composition for the eyelashes may be performed immediately after applying mascara;
the application of a skincare composition may be performed immediately after applying a cleansing solution.

The compositions may be applied daily or at a frequency of 2 to 3 applications per week or more, for a duration of 1 to 3 months or more, which is renewable depending on the degree of impairment-of the individual's keratin substrates to be treated.

In particular, the process according to the invention will be intended for caring for, treating, strengthening and/or repairing sensitized keratin substrates, and preferentially sensitized hair.

The term “sensitized hair” means hair that has been embrittled or damaged by hair treatments, for instance relaxing, permanent-waving or dyeing operations, or by the effect of atmospheric agents. Sensitized hair is coarse after washing and drying, has poor mechanical behaviour, more static electricity and looks dull.

The invention also relates to the cosmetic use of at least one polypeptide of the KAP family in a composition, as an agent for maintaining and/or restoring and/or improving the protein content of a keratin substrate.

In particular, the polypeptide of the KAP family will be intended and used especially for:
maintaining and/or restoring and/or improving the protein content of a keratin substrate;
maintaining and/or restoring and/or improving the surface state of the keratin substrates;
conserving and/or restoring and/or improving the physical and/or mechanical properties of the keratin substrates.

The agent will thus be intended and used especially for improving the softness of keratin fibres, and/or their suppleness and/or elasticity and/or resistance to breaking and/or volume and/or hairstyle hold and/or resistance to chemical treatments.

The compositions according to the invention will thus be intended and used to improve the appearance and/or surface state of keratin substrates, in particular to make the hair softer, smoother, less split and more resistant to breaking, to give it greater consistency and body, especially more volume and more hairstyle hold, more elasticity and suppleness, and also greater resistance to treatments liable to weaken it (e.g.: dyeing or permanent-waving).

The invention is illustrated in greater detail in the non-limiting examples that follow.

### EXAMPLES

The values are expressed as percentages relative to the total weight of the composition, unless otherwise indicated.

These formulations are prepared according to known methods.

Shampoo

| | |
|---|---|
| Fragment SPCCR of sequence SEQ ID No. 9 | 1% |
| Sodium lauryl ether Sulfate | 15% |
| Purified water | qs 100% |

Mascara

| | | |
|---|---|---|
| Fragment SPCCR of sequence SEQ ID No. 9 | 1 | g |
| Carnauba wax | 20 | g |
| Polyoxyethylenated (30 OE) glyceryl Stearate | 8 | g |
| (Tagat S from the company Goldschmidt) | | |
| Black iron oxide | 5 | g |
| Propylene glycol | 5 | g |
| Hydroxyethylcellulose | 2.5 | g |
| Water | qs 100 | g |

The invention compositions and methods are preferably used by subjects desirous of the benefits noted herein, subjects “in need of” these benefits. One using the invention as disclosed will preferably use an amount of the invention composition effective to produce the desired benefit(s). Such amount is inclusive of an amount of the compositions described herein at the disclosed concentrations of active ingredients sufficient to cover the keratin being treated in a single application, and also includes an amount applied upon repeated application, for example on a daily basis over a course of days, weeks, etc. In a preferred embodiment the invention process includes multiple applications of the invention composition to keratin materials in need of attention.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials. Terms such as “contain(s)” and the like as used herein are open terms meaning including at least unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles, defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium suitable for topical application to keratin substrates, at least one KAP polypeptide and at least one cosmetic ingredient chosen from keratin conditioning agents, care agents, :treating agents, and mixtures thereof.

2. The composition according to claim 1, comprising at least one keratin conditioning agent chosen from oils, waxes, ceramides, gums, silicones, polysiloxanes, film-forming polymers, fixing polymers, and mixtures thereof.

3. The composition according to claim 1, comprising at least one agent chosen from moisturizers, agents for stimulating the synthesis of dermal or epidermal macromolecules and/or for preventing their degradation, softeners, free-radical scavengers, vitamins, amino acids, proteins, antidandruff agents, anti-seborrhoeic agents, reducing agents, hair dye precursors, direct hair dyes, oxidizing agents, and mixtures thereof.

4. The composition according to claim 1, wherein the cosmetic ingredient(s) is(are) present in the composition in an amount ranging from 0.001% to 20% by weight relative to the total weight of the composition.

5. The composition according to claim 1, wherein it further comprises at least one cosmetic adjuvant or formulating agent chosen from organic solvents, surfactants, plasticizers, thickeners, emulsifiers, preserving agents, UV-screening agents, fillers, dyestuffs, fragrances, opacifiers, humectants, agents for adjusting and fixing the pH, antibacterial agents, antifungal agents, propellants, and mixtures thereof.

6. The composition according to claim 1, wherein the at least one KAP polypeptide comprises at least one peptide sequence chosen from (i) the sequences SEQ ID No. 1 to SEQ ID No. 66, (ii) a fragment of the sequences comprising at least 3 consecutive amino acids of a repeating sequence chosen from the sequence SZCCXPSCCXP (Z: Ø, P and X: Q,V,R,I) and the sequence YGGXGYGSGY (X:Y,L,F) and (iii.) homologues thereof.

7. Composition according to claim 6, comprising at least one peptide fragment (ii) comprising from 3 to 60 amino acids.

8. The composition according to claim 1, wherein the polypeptide of the KAP family is of natural origin.

9. The composition according to Claim 1, wherein the KAP polypeptide is present in the composition in an amount of fom 0.001% to 30% by weight relative to the total weight of the composition.

10. The composition according to claim 1, wherein said composition is in the form of aqueous, aqueous-alcoholic or oily solutions, oil-in-water (O/W), water-in-oil (W/O) or multiple (W/O/W or O/W/O) emulsions, aqueous or oily gels, dehydrated anhydrous products, or dispersions of an oily phase in an aqueous phase having nanoparticle spherules or ionic and/or nonionic lipid vesicles.

11. The composition according to claim 1, wherein said composition is a haircare lotion, a shampoo or a hair conditioner, a liquid or solid cleansing soap, a hairstyle shaping product, a colouring shampoo, a dye, a permanent-waving composition, a treating mask, a cream, a foaming cleansing gel, a mascara for making up the eyelashes, a base for making up the eyelashes, a top coat composition to be applied over a mascara, a cosmetic treatment composition for the eyelashes, a nail varnish, a nail base or “base coat”, a finishing composition or “top coat”, a nail varnish dissolver, a treating base for the nails, a cream, an ointment, a milk, a lotion, a serum, a paste, a mousse or a stick.

12. A process wherein a composition as defined in claim 1 is applied to the skin, the hair, the eyelashes or the nails, optionally followed by rinsing.

13. The process according to claim 12, wherein the composition is applied as a pre-treatment during a treatment liable to sensitize the skin, the hair, the eyelashes or the nails, and/or as a post-treatment.

14. A process according to claim 12, wherein the composition is applied to at least one of sensitized skin, hair, eyelashes or nails.

15. A process, comprising applying an amount of a composition of claim 1 to a keratin substrate effective to maintain and/or restore and/or improve the protein content thereof.

16. A process, comprising applying an amount of a composition of claim 1 to a keratin substrate effective to conserve and/or restore and/or improve the physical and/or mechanical properties thereof.

17. A process, comprising applying an amount of a composition of claim 1 to a keratin substrate effective to maintain and/or restore and/or improve the surface state of the keratin substrates.

18. A process, comprising applying an amount of a composition of claim 1 to a keratin substrate effective to improve the softness and/or suppleness and/or elasticity and/or resistance to breaking and/or volume and/or hold and/or resistance to chemical treatments thereof.
